(19) United States
(12) Patent Application Publication
(10) Pub. No.: US 2008/0300451 A1
(43) Pub. Date: Dec. 4, 2008

(54) Method for Placing a Jacket on a Section of a Natural Vessel with the Aid of a Tubular Auxiliary Instrument

(76) Inventors: Thomas Beck, Durchhausen (DE); Dennis Langanke, Tuttlingen (DE); Anton Moritz, Frankfurt (DE)

Correspondence Address: NATH & ASSOCIATES, 112 South West Street, Alexandria, VA, 22314, US

(21) Appl. No.: 11/660,278

(22) PCT Filed: Aug. 16, 2005
(86) PCT No.: PCT/EP05/08854

§ 371 Date: Aug. 17, 2007

(30) Foreign Application Priority Data
Aug. 19, 2004 (DE) 10 2004 041 067.4

(51) Int. Cl. A61F 2/06; A61B 17/00

(52) U.S. Cl. 600/36; 606/153

(57) ABSTRACT

The invention relates to a method for placing a flexible jacket 3 on a natural vessel section 5, in particular a vein, to a device for placing a flexible jacket 3 in accordance with said method, and to an auxiliary instrument 1 for use in said method. The method comprises the following steps:
pushing the jacket 3 onto a tubular application aid 2,
filling the vessel section with a fluid,
introducing, in particular pulling, the filled vessel section 5 into the application aid 2, the filling of the vessel section and the introduction of the vessel section 5 into the application aid 2 being carried out with the assistance of a tubular auxiliary instrument 1, and
pulling the application aid 2 and the tubular auxiliary instrument 1 apart while at the same time pulling the vessel section 5 out of the application aid 2 and depositing the jacket 3 on the surface of the vessel section 5.

The invention relates to a method for placing a flexible jacket on a natural vessel section, in particular a vein, to a device for placing a flexible jacket in accordance with said method, and to an auxiliary instrument for use in said method.

For treatment of vascular diseases caused by changes to blood vessels, for example arteriosclerosis, modern surgery often makes use of so-called bypasses in the form of vessel replacement implants. Biological vascular materials of natural origin are often used for this purpose, in order to avoid undesired reactions and interactions in the recipient organism, such as the formation of clots, caused by non-biological materials, for example metallic or fully synthetic materials. These natural vessel replacement implants are generally obtained from veins. To ensure that they meet the special requirements for permanent and stable arterial replacement vessels, in particular for coronary bypass, their stability has to be strengthened and their outer shape adapted in order to withstand the high internal pressure of the arterial vessels.

Such strengthening and adaptation can be achieved, for example, by means of a shaping and stabilizing jacket being pulled onto the vessel, in particular the vein. Thus, according to DE 199 10 340, a hose, a sheath or a tube is used as a jacket for a vein for artery replacement transplants. An example of a tubular jacket for a vein, with a mesh-like structure, is to be found in DE 101 37 414 A1.

To make it easier to pull a jacket onto the natural vessel, and at the same time to avoid the danger of damaging the outer wall of the vessel, the jacket can first of all be drawn onto a tubular application aid. The vessel is then inserted into the application aid, after which the latter is removed again, with the jacket at the same time being deposited onto the surface of the vessel.

However, a fundamental problem that arises when applying the jacket by means of a tubular application aid is that the unstabilized and extremely flexible natural vessel section, which is highly sensitive and susceptible to damage, has to be pulled into the tubular application aid and then pulled completely right through the latter without the vessel, and in particular the outer walls, being impaired or damaged in the process. Moreover, a further problem often arises when the jacket is deposited onto the vessel. Because of the soft and unstable consistency of a natural vessel, such as a vein explant, it is often very difficult to deposit the jacket onto the surface of the vessel in uniform manner and in particular without folds forming. Of course, the vessel section also has to be free of kinks or folds even in the jacketed state.

Ann Thorac Surg 1994; 57; 240-2 describes a vein being pulled into a tubular application aid by means of a thread which is knotted onto one end of the vein and which, after the vein has been completely pulled in, is cut off together with the end of the vein. However, this entails the problem of insertion of the thread through the application aid, especially in the case of longer vein explants.

Ann Thorac Surg 1992; 127; 416-419 describes the use of a mesh-like, porous and flexible jacket for stabilizing a natural vein explant obtained from varicose veins, and for correcting and adapting its outer form. To generally examine whether varicose veins are suitable as vessel replacements, vein sections are filled with X-ray contrast medium and then with wax and are cut into short pieces for evaluation.

In U.S. Pat. No. 4,743,251, FIG. 2B shows a thin rod onto which a vein end is knotted. The secured vein is subsequently pulled into and through an application aid by means of the rod. The rod is then cut off with the knotted end of the vein.

However, both approaches, that is to say insertion of a vein into the application aid with a rod and also by means of a knotted thread, have the crucial disadvantage that the vascular tissue is subjected to very high loads by the extremely fine, tear-resistant and therefore very sharp thread material in the area of the knot. This can easily lead to severing of the vessel section, particularly in the case of varicose veins, immediately during the secure knotting and, in particular, inside the application aid while being pulled into the jacket, because of the tensile forces that arise exactly at the point of knotting. Moreover, the securing or knotting represents an additional, time-consuming step during the operation.

DE 102 32 134 A1 from the Applicant describes an auxiliary instrument for pulling a vessel section into an application aid, said auxiliary instrument having the form of a rod-shaped gripper. By using such a gripper, it is possible to manage without additional securing means, thereby avoiding the disadvantages described above in connection with the knotting of the vessel. However, the problem of fold formation when the jacket is deposited on the vessel section still remains.

It may therefore be stated that the prior art has not yet disclosed a comprehensive and satisfactory solution to the problem of placing a jacket on a vessel section, particularly in terms of the fold formation that occurs when depositing the jacket onto the vessel section.

Therefore, the object of the invention is to make available a method with which a flexible jacket is placed on a vessel section and which overcomes the problems of the prior art. In particular, the method is intended to allow the vessel to be pulled safely, quickly, easily and gently into an application aid, in conjunction with fold-free deposition of the jacket on the surface of the vessel.

According to the present invention, this object is achieved by a method for placing a flexible jacket on a natural vessel section, in particular a vein, said method comprising the following steps:
pushing the jacket onto a tubular application aid,
filling the vessel section with a fluid,
introducing, in particular pulling, the filled vessel section into the application aid, the filling of the vessel section and the introduction of the vessel section into the application aid being carried out with the assistance of a tubular auxiliary instrument, and
pulling the application aid and the tubular auxiliary instrument apart while at the same time pulling the vessel section out of the application aid and depositing the jacket on the surface of the vessel section.

By filling the vessel section with the fluid, it is possible for a vessel section of soft, unstabilized consistency to be converted into an at least partially stabilized form, if appropriate even an expanded form, with a comparatively tight and fold-free surface. In this stabilized form, the introduction of the filled vessel section into the application aid is made easier, particularly in respect of the fold-free deposition of the jacket on the surface of the filled vessel, and this provides considerable advantages over conventional approaches.

By using the tubular auxiliary instrument both when pulling the vessel section into the application aid and also when filling it, simple and quick implementation of the method is ensured. The degree of filling of the vessel section can be adjusted or corrected, via the tubular auxiliary instrument, immediately before or during the pulling-in of the vessel section. The individual method steps are described in more detail below.

The auxiliary instrument preferably used can be described as a straight, preferably flexurally stiff tube with a through-channel, preferably a continuous through-channel. In particular, an auxiliary instrument is used which is designed in the manner of a cannula.

In a preferred embodiment of the method according to the invention, the vessel section to be filled and to be introduced into the application aid is first of all connected to a first end of the tubular auxiliary instrument. The vessel section is especially preferably fitted onto the end. The fitted vessel section can optionally be secured, in particular clipped or tied, for example by mechanical means, against sliding out of place or slipping off the end of the auxiliary instrument. Adhesive attachment of the vessel section is also possible. Conventional suture material known to a person skilled in the art can be used for binding it.

The filling of the vessel section is preferably carried out by means of a filling device, especially by means of a syringe, in particular a syringe barrel with plunger. For this purpose, the filling device is connected to a second end of the tubular auxiliary instrument. By way of the auxiliary instrument, a vessel section secured on its first end can thus be filled with the fluid. Particularly advantageously, the vessel section can also be flushed with the fluid, before being finally filled. A check can also be made for leaktightness, which can be especially important if one or more ligatures are present. For this purpose, the vein is closed off at its free end.

All free-flowing media of a liquid or gaseous type can in principle be used as the fluid. However, liquids, in particular aqueous solutions, e.g. physiological saline solution, are preferably used for flushing and filling.

In a particularly preferred embodiment of the method according to the invention, the jacket is first of all pushed onto the tubular application aid. To do so, the end of the tubular application aid onto which the jacket is pushed can be provided in a known manner with a stopper, with a push-on cone or the like. As has already been described, the vessel section that is to be jacketed is then connected to the first end of the tubular auxiliary instrument. Thereafter, the auxiliary instrument is preferably guided through the tubular application aid such that the vessel section connected to the tubular auxiliary instrument is not yet inserted into the application aid, but the second end of the auxiliary instrument already emerges from the latter. The second end of the auxiliary instrument is then preferably connected to the filling device for filling the vessel. It may equally be preferable for the tubular auxiliary instrument to be first guided through the application aid (or to be already located in it) and for its first end then to be connected to the vessel section and its second end to the filling device. Before filling takes place, the vessel section may optionally be flushed with the fluid, and a check can be made to ensure leaktightness. In the next step, the vessel section is preferably filled via the tubular auxiliary instrument and is then drawn into the application aid by pulling on the auxiliary instrument. Alternatively, however, these two steps can also be carried out in the reverse order. Finally, the application aid and the tubular auxiliary instrument are pulled apart, with the vessel section at the same time being pulled out of the application aid and, at the same time, the jacket being deposited on the outer face of the vessel section.

The natural vessel sections to be provided with a jacket by the method according to the invention are preferably blood vessel sections, preferably parts of veins, in particular varicose veins. Veins that have been provided with a jacket can be stored in the known way until they are to be used.

The present invention also relates to a device for placing a flexible jacket on a natural vessel section in accordance with a method according to the invention. The device according to the invention comprises a tubular auxiliary instrument with a through-channel, in particular in the manner of a cannula, a tubular application aid into which the tubular auxiliary instrument can be inserted, and/or a flexible jacket that can be pushed onto the tubular application aid.

The application aid is expediently of such length that it is able to completely receive the jacket, at least in the contracted state.

The tubular auxiliary instrument is preferably longer than the application aid. It is also preferable that the tubular auxiliary instrument, along its full length, has an external diameter smaller than the internal diameter of the application aid, such that it can be guided through the application aid together with the vessel section.

Preferably, the application aid has essentially the shape of a cylindrical tube. It is preferably widened in a funnel shape at one end in order to facilitate insertion of the auxiliary instrument and of the vessel section at this end. If appropriate, the application aid comprises a conical stopper for fitting onto its other end. This allows the jacket to be pushed onto the application aid more easily and more gently. Once this has been done, the conical stopper has fulfilled its purpose and can be removed again. It is also possible, in principle, to push the attached stopper out using the auxiliary instrument when the latter is inserted into the application aid.

The inner surface of the application aid is preferably smooth and free of irregularities, such that the method according to the invention ensures that the vessel section is pulled in without sustaining damage.

In principle, the material composition of the application aid is not critical. According to one embodiment, at least part of the application aid is made of metallic materials, preferably of stainless and cold-hammered steel. It is also possible for the application aid to be made from non-metallic materials, preferably of plastics, in particular PPA, PEEK or LCP (liquid crystal polymer).

In a particular embodiment of the device according to the invention, the jacket can already be arranged on the application aid. The jacket is preferably already produced in a tubular shape and is preferably designed as described in WO 03/011190 A2, to whose content reference is hereby made.

Correspondingly, provision can also be made for the auxiliary instrument to be already arranged inside the application aid. It is also possible for the device to contain further components, for example suture material, e.g. in the form of a kit.

A person skilled in the art will appreciate that the application aid, and the other components of the device too, can be sterilized by a suitable sterilization method using customary chemical or physical methods, and in particular can be packaged in sterile form.

The tubular auxiliary instrument used in the method according to the invention also forms part of the subject matter of this invention. As has already been mentioned, it is of particular advantage, as regards the practical implementation of the described method according to the invention, that two such different individual steps such as filling and pulling-in of the vessel can be carried out using the same instrument.

According to the invention, a tubular auxiliary instrument for use in the method according to the invention has a through-channel and is designed in particular in the manner of a cannula. It mainly comprises a tubular portion with a first end and a second end. The first end of the tubular portion is advantageously configured in such a way that it provides a fitted vessel section with a secure hold against sliding out of place or slipping off. A connector piece for securing a filling device, in particular a syringe, is preferably provided on the second end of the tubular portion.

The first end of the tubular portion preferably has a retaining means designed in particular as at least one annular enlargement. The latter preferably extends transverse to the longitudinal axis of the tubular portion, around the outer circumference of the latter. The retaining means is particularly preferably designed in the shape of an olive. Toward the outside, the end can be conically shaped to allow a vein to be easily pushed on. Alternatively, the retaining means can also involve a profiling of the surface of the first end of the tubular portion.

In a particularly preferred embodiment of the auxiliary instrument according to the invention, the connector piece for securing the filling device is an adapter. In the case of a syringe being used as the filling device, the connector piece is in particular a Luer lock adapter.

The auxiliary instrument can be a disposable product, or a reusable product that can be sterilized after use. The auxiliary instrument is preferably made of the same materials that can be considered for producing the application aid, in particular metal materials, such as stainless, cold-hammered steel, or plastic, for example PPA, PEEK or LCP.

The present invention also relates to the use of the tubular auxiliary instrument according to the invention for placing a flexible jacket on a natural vessel section, in particular for pulling the vessel section into a tubular application aid supporting the jacket.

The present invention, and in particular the way it works, will be described below through a detailed description of particular embodiments. In these embodiments, individual features of the invention can be implemented singly or in combination with other features. The particular embodiments described here serve only to explain and to provide a better understanding of the invention, and they are not in any way to be understood as limiting the invention.

### DESCRIPTION OF THE FIGURE

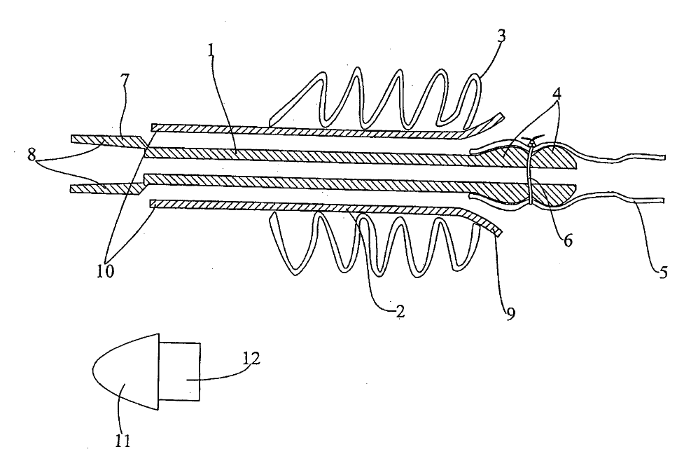

In the drawing, FIG. 1 shows a longitudinal section through an embodiment of the device according to the invention for placing a flexible jacket on a vessel section. A cannula 1 as tubular auxiliary instrument, which is used for inserting a vessel section, in particular a vein, into a mesh-like jacket, is surrounded by an application aid designed as tube 2. On its outside, the tube 2 supports the mesh-like jacket 3 which is intended to be placed on the vessel section. The jacket 3 is shown schematically.

The first end of the cannula 1 has two annular enlargements 4 serving as retaining means for securing the vessel section 5 (also shown schematically), which is ligated onto this end of the cannula. In the area between the two enlargements 4, the vessel section 5 is secured by a thread 6 against sliding out of place or slipping off from the end. An adapter 7 for attachment of a syringe is formed integrally onto the second end of the cannula 1. The adapter 7 has an inner cone 8 into which an outer cone of a syringe barrel can be fitted.

The tube 2 serving as application aid mainly has a cylindrical shape with a circular cross section. The internal diameter of the tube 2 is equal to or greater than the maximum external diameter of the cannula 1 and equal to or greater than that of the vessel section 5 to be pulled through the tube 2. The tube 2 is widened in a funnel shape at one end 9. The cannula 1 can be easily inserted at this end 9. A conically shaped stopper 11 can be fitted onto its second end 10. When the stopper 11 is fitted, the jacket 3 can easily be applied to the tube 2. The stopper is provided with an abutment shoulder 12 with which it comes to bear on the front face of the end 10. Special retaining means can also be provided.

The length of the tube 2 can be between 4 cm and 20 cm, preferably between 7 cm and 15 cm. Its diameter is preferably between 5 mm and 12 mm. The wall thickness of the tube 2 is advantageously less than 0.5 mm, in particular 0.2 mm to 0.3 mm. The tube 2 has a preferred internal diameter of between 4.5 mm and 11.5 mm.

The cannula 1 is longer than the tube 2. Its length is preferably between 8 cm and 16 cm. It has an external diameter of between 4.5 mm and 10 mm. The actual value is in each case dependent on the internal diameter of the tube 2 (or vice versa) and can vary along the cannula 1. The cannula 1 preferably has its greatest diameter in the area of the adapter 7. The diameter of the cannula 1 is preferably smaller in the area of the enlargements 4. The internal diameter of the cannula 1 should not be less than 1 mm.

To place a mesh-like jacket 3 on a vessel section 5 using the device shown in longitudinal section, the vessel section 5 on which the jacket is to be placed is first fitted onto the first end of the cannula 1. For this purpose, the vessel section 5 is pushed over the two annular enlargements 4. If appropriate, the vessel section 5, lying between the two enlargements 4, is additionally secured by a thread 6 against slipping off. Thereafter, a syringe is attached to the adapter 7. The vessel section 5 can then be flushed with physiological saline solution, for example. If any ligatures have been placed on side branches, their leaktightness can also be checked by means of the vessel section 5 being expanded by injection of the solution under pressure. For this purpose, the vessel section 5 is closed at its free end. If it has not already been done, the conically shaped stopper 11 is fitted onto the second end 10 of the tube 2. The jacket 3 is pushed onto the tube 2, whereupon the stopper 11 can be removed again. To pull the vessel section 5 into the tube 2, any syringe that has been attached is withdrawn from the adapter 7, and the cannula 1 is pushed, with the adapter 7 in the lead, through the funnel-shaped widened end 9 and through the tube 2, until the adapter 7 can be gripped at the other end 10 of the tube 2. The syringe can now be attached again. It is also possible to use the syringe as a handpiece for the attached cannula 1. In this way, the cannula can be pushed from the second end 10 of the tube into the latter, after the jacket has already been pushed onto the tube 2. Only then is the vessel section 5 secured on the first end or the enlargements 4 of the cannula 1 and then filled, if appropriate after being flushed. By pulling on the cannula, the vessel section 5 can now be drawn into the tube 2 serving as application aid and can subsequently be withdrawn from the latter. Upon withdrawal, the jacket 3 is deposited uniformly on the outer face of the vessel section 5. With the syringe attached, the degree of filling of the vessel section 5 can be adjusted or corrected directly before or during the pulling-in. When the jacket 3 has been applied, the jacketed vessel section 5 can be cut off or withdrawn from the end of the cannula 1 and can then be cut to the required length.

1-15. (canceled)

16. A method for placing a flexible jacket on a natural vessel section, said method comprising the following steps:
pushing the jacket onto a tubular application aid,
filling the vessel section with a fluid,
introducing the filled vessel section into the application aid, the filling of the vessel section and the introduction of the vessel section into the application aid being carried out with the assistance of a tubular auxiliary instrument, and
pulling the application aid and the tubular auxiliary instrument apart while at the same time pulling the vessel section out of the application aid and depositing the jacket on the surface of the vessel section.

17. The method as claimed in claim 16, wherein a tube with a through-channel is used as auxiliary instrument.

18. The method as claimed in claim 16, wherein the vessel section to be filled and to be introduced into the application aid is connected to a first end of the tubular auxiliary instrument.

19. The method as claimed in claim 16, wherein the filling is carried out by means of a filling device which is connected to a second end of the tubular auxiliary instrument.

20. The method as claimed in claim 16, wherein a liquid is used as fluid.

21. The method as claimed in claim 16, comprising the following steps:
pushing the jacket onto a tubular application aid,
connecting the vessel section to the first end of the tubular auxiliary instrument,
guiding the tubular auxiliary instrument through the application aid,
connecting the second end of the auxiliary instrument to the filling device,
optionally flushing the vessel section with the fluid, filling the vessel section with the fluid via the tubular auxiliary instrument,
pulling the filled vessel section into the application aid by pulling on the auxiliary instrument, and
pulling the tubular auxiliary instrument and the application aid apart while at the same time pulling the vessel section out of the application aid and at the same time depositing the jacket on the outer face of the vessel section.

22. A device for placing a flexible jacket on a natural vessel section in accordance with the method as claimed in claim 16, said device comprising a tubular auxiliary instrument with a through-channel, and at least one member of the group consisting of a tubular application aid into which the tubular auxiliary instrument can be inserted, and a flexible jacket that can be pushed onto the tubular application aid.

23. The device as claimed in claim 22, wherein the application aid is of such length that it is able to completely receive the jacket, if appropriate in the contracted state.

24. The device as claimed in claim 22, wherein the tubular auxiliary instrument is longer than the application aid.

25. The device as claimed in claim 22, wherein the jacket is located on the application aid.

26. The device as claimed in claim 22, wherein the tubular auxiliary instrument can be guided through the application aid.

27. The device as claimed in claim 22, which comprises a tubular portion with a first end and a second end, the first end of the tubular portion being configured in such a way that it provides a vessel section fitted onto the first end with a secure hold against sliding out of place or slipping off, and a connector piece for securing a filling device on the second end of the tubular portion.

28. The device as claimed in claim 27, wherein the first end has at least one retaining means.

29. The device as claimed in claim 27, wherein the connector piece is an adapter for securing the filling device.

30. The device as claimed in claim 22, which is used for placing a flexible jacket on a natural vessel section.

31. A method as claimed in claim 16, wherein the natural vessel section is a vein.

32. A method as claimed in claim 17, wherein the through-channel is in the manner of a cannula.

33. A method as claimed in claim 19, wherein the filling device is a syringe.

34. A method as claimed in claim 20, wherein the liquid is an aqueous solution.

35. A device as claimed in claim 22, wherein the through-channel is in the manner of a cannula.

36. A device as claimed in claim 27, wherein the filling device is a syringe.

37. A device as claimed in claim 28, wherein the retaining means is an annular enlargement.

38. A device as claimed in claim 28, wherein the retaining means extends traverse to the longitudinal axis of the tubular portion around the outer circumference of the latter.
